(19) United States
(12) Patent Application Publication
(10) Pub. No.: US 2016/0361596 A1
(43) Pub. Date: Dec. 15, 2016

(54) PORTABLE WALL-ADHERING EXERCISE AID

(71) Applicants: Iberia Grace Urquiza Brogmus, Burbank, CA (US); George Erich Brogmus, Burbank, CA (US)

(72) Inventors: Iberia Grace Urquiza Brogmus, Burbank, CA (US); George Erich Brogmus, Burbank, CA (US)

(21) Appl. No.: 14/737,346

(22) Filed: Jun. 11, 2015

(51) Int. Cl. A63B 24/00; A61B 5/11; A61B 5/01

(52) U.S. Cl. CPC A63B 24/0062; A61B 5/01; A61B 5/11

(57) ABSTRACT

A portable wall-adhering exercise aid is provided. In an implementation, the exercise aid includes a sheeted material that removably adheres to a wall or other surface and provides a low-friction surface for exercise support. The exercise aid can provide balance to a user during exercises, such as back exercises, and can bear some of the weight during exercise making the exercise session more attractive. The exercise aid may include pressure sensors and heart sensors. An embedded processor may provide real time feedback and provide programmed exercise routines. The processor and a transceiver may also enable remote control or establish telemetry for remote monitoring.

### BACKGROUND

There is a plethora of exercise equipment. Exercise equipment ranges from highly specialized systems that target specific muscle groups to equipment that can be used for a variety of exercise purposes, such as free weights. The spectrum of exercise equipment by target use ranges from aerobic-focused equipment, such as treadmills, stair-steppers and elliptical to anaerobic-focused equipment intended to build muscle strength, mass and/or definition.

The health benefits of exercise are well-proven to prolong life, delay the onset of age-related declines in performance and health, and improve well-being. Exercise can also be particularly beneficial to individuals with certain conditions as part of recovery or for delaying or compensating for physical abilities lost due to the ailment. For example exercise has been shown to improve balance in the elderly and in Parkinson's sufferers. Increased exercise is also prescribed against the growing concern of obesity, and especially obesity in children.

Yet many individuals have reasons for not engaging in traditional or equipment-enhanced exercise. One such reason is lack of balance for the aging and disabled or lack of coordination for the very young. With a growing aging population, there are more individuals struggling with age-related balance maladies as well as balance pathologies related to diseases, disorders, and prescription or non-perscription drug use. Another reason for not engaging in exercise is that many exercises are too painful to do when experiencing back pain or other musculoskeletal pain. Back pain is the leading cause of worker disability, is experienced by a large portion of the population each year, and is a leading reason for seeking medical care. Low back pain causes more global disability that any other condition. Back pain is also the primary reason for seeking alternative medical treatments and complementary care. Despite the pain, recovery is better the more active one remains, and exercise that promotes the requisite activity without undo back stress would be beneficial. Individuals also avoid exercise for various other reasons: lack of strength to begin a weight-intensive exercise program, lack of time or space to exercise, reticence to leave home, and aversion to gyms. Many people do not like to visit a public gym when traveling, but would like to have supported exercise wherever they go, with gym capability that they can bring along with them.

### SUMMARY

A portable wall-adhering exercise aid is provided. In an implementation, the exercise aid includes a sheeted material that removably adheres to a wall or other surface, without damage, marring or leaving residue, and provides a low-friction surface for exercise support. One or more tabs or other physical components, connected to the surface, can be pinched or grasped to pull the surface from the wall easily. The exercise aid can provide balance to a user during exercises, such as back exercises, and can bear some of the weight during exercise making the exercise session more attractive and less biomechanically stressful than unsupported exercise. The exercise aid may include pressure sensors and heart sensors. An embedded processor may provide real time feedback and provide programmed exercise routines. The processor and a transceiver may also enable remote control or establish telemetry for remote monitoring.

This summary section is not intended to give a full description of portable wall-adhering exercise aids. A detailed description with example implementations follows.

### BRIEF DESCRIPTION OF THE DRAWINGS

FIG. 1 is a diagram of an example exercise aid.

FIG. 2 is a diagram of an example remote control unit associated with the example exercise aid.

FIG. 3 is a diagram of an example use of the example exercise aid.

FIG. 4 is a diagram of an example alternative implementation of the example exercise aid.

FIG. 5 is a diagram of another example alternative implementation of the example exercise aid.

FIG. 6 is a block diagram of example computing components that may be used in implementations of the example exercise aid.

### DETAILED DESCRIPTION

### Overview

This disclosure describes example portable wall-adhering exercise aids. An example implementation provides portable, low-biomechanical-stress exercise equipment. The biomechanical stress experienced when using the example exercise aid is low relative to that of similar exercises for which no support is provided.

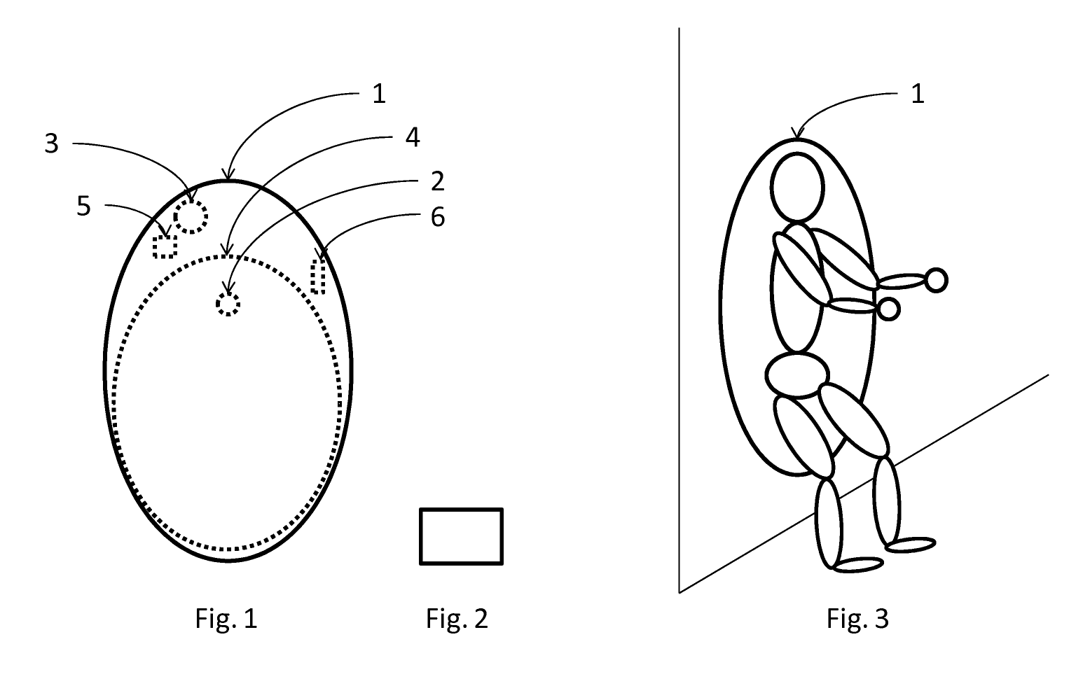

FIG. 1 shows an example exercise aid 1. In an implementation, the exercise aid 1 includes a heart rate sensor 2, an audio device, such as a speaker 3 or other headphone element or cell phone element, a pressure sensor array 4 (e.g., thin-film transducers), a processing unit 5, including for example, a microprocessor with supporting components, a transmitter (or transceiver) for communicating with a remote (FIG. 2), and a tab or other physical component(s) 6 to facilitate removal. The said remote may be a known device. Examples of known remotes include smartphones, tablets (including iPads and other operating-system-based systems), gaming systems (e.g., Playstation, Wi, Xbox, etc.) and their controller, and appliance remotes.

In an implementation, the example exercise aid 1 is self-adhering (e.g., to a wall) and may function as a protector for the wall or other surface to facilitate standing (or other posture) exercises utilizing the wall or surface to supplement a user's own intrinsic support and balance during exercise. Alternatively, the exercise aid 1 may be fastened to a wall or surface more permanently with additional fastening hardware, such as screws, bolts, nails, molly bolts, tapes, staples, adhesives, etc. Said fastening may include borders that can be shaped and surfaced to not interfere with the intended contact area.

FIG. 3 shows an example use scenario of the example exercise aid 1. The exercise aid 1 can be a self-adhering sheet or a sheet the adheres through static cling, such as vinyl or other suitable pliable, low-friction flexible or semi-flexible sheeted material that remains on a surface during exercises in which the user presses body areas (e.g., bottom and back) against the exercise aid 1 to supplement the user's support and balance. An example exercise aid system may include audio or video suggestions on standing exercises intended to provide the benefits of calisthenics (but without the need to get onto the floor, for example, and without high-impact stresses to the body). The medium used to communicate the audio or video suggestions may include paper or electronic techniques, such as built-in speaker(s), transmission to a smartphone/tablet/bluetooth speaker, headphones, earphones, television, computer, etc.

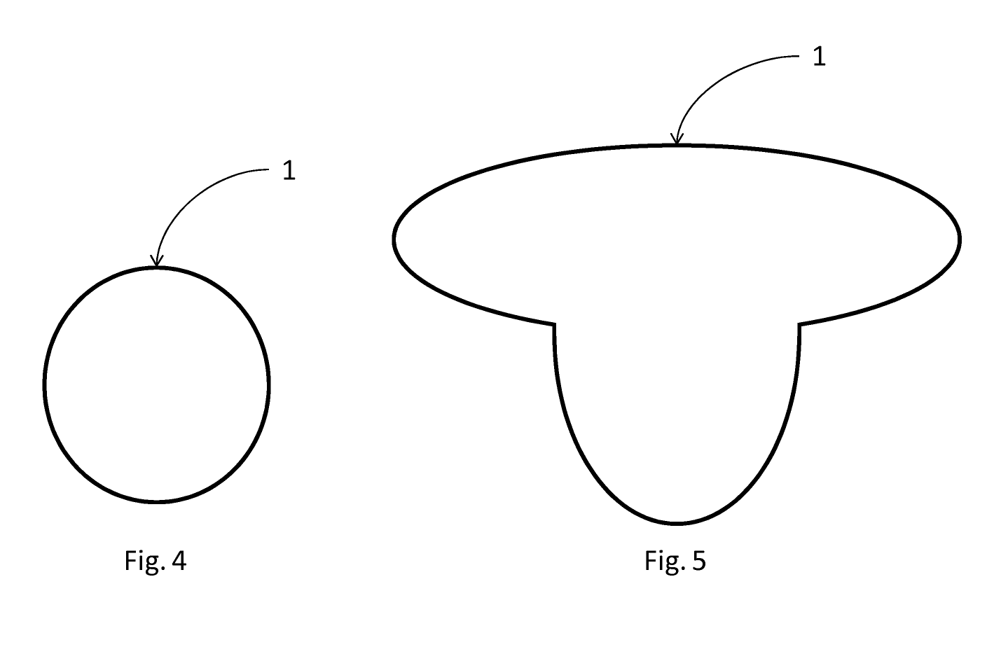

The example exercise aid 1 may have a variety of shapes and sizes. FIG. 4 shows an alternative shape of the exercise aid 1 for supporting the user mainly through contact with the user's bottom. FIG. 5 shows another alternative (2-dimensional) configuration to support the user (and protect a wall or surface) while accommodating contact with the user's outstretched arms and hands. The exercise aid 1 may assume various shapes and configurations depending on the user's desired contact area with the wall or surface (e.g., just a bottom area of the body; just bottom, back, and shoulders; or entire upper body including arms and hands, etc.).

In an example scenario, the exercise aid 1 serves to:
Provide a low-friction surface for exercising against a wall;
Protect the wall from frictional damage or from getting soiled or dirty; and
Provide support to improve balance during exercise.

The example exercise aid 1 may also allow a user to stay at home, but is also easily portable so that the exercise aid 1 can be taken along during trips or travel. When used on a vertical surface, the example exercise aid 1 requires no horizontal floor space and essentially has a zero-footprint with respect to floor space needed beyond the space taken by the user. The exercise aid 1 may enable weak individuals (or individuals suffering from back pain or other musculoskeletal pain) to exercise at their own pace (the time they want to spend and the intensity with which they wish to exercise), all within their current capabilities.

In addition to different sizes and shapes, for example those shown in FIGS. 4-5, the example exercise aid 1 may also include electronic monitoring and feedback features. These may include audible or visible exercise routines or pacing, pressure sensing, heart rate, blood-oxygen, etc., monitoring, and/or repetition, speed, and frequency information. Other electronic features may include clock, radio, video projection and/or smart-device application to give added versatility. These features may be included as one or more light-weight electronic panels integrated into the sheeted material of the example exercise aid 1, with one or more interfaces, such as video or game connections and telemetry, or may be implemented as remote or stand-alone components. Pressure sensors, such as a pressure-responsive array 4 may be embedded in, or applied to, the sheeted material to provide pressure readings for analysis of location and peak values for estimating, e.g., frequency of repetitions. A heart rate sensor 4 for sensing cardiac parameters (and blood-oxygen level, etc.) can be implemented through an optional wrist-worn heart-rate monitor, a (finger) heart/oxygen monitor, a microphone built into the sheeted material (that senses heart rate as the user is pressed up against the exercise aid 1), or a smart-device application that uses current technologies to remotely sense heart rate, e.g., via camera or sonic pick-up.

The example exercise aid 1 may be customizable so that companies can place their own wording, design, logo, photograph(s), or images onto the visible surface as a promotional item or employee reward. The adhesive and electronics may even be used solely as an advertising feature as well without utilization of the exercise utility. Individuals may also customize the exercise aid with their own photos or graphics.

Likewise, the example exercise aid 1 can be used to encourage children to exercise. For younger children, a custom graphic may include their favorite character, with approved consent when using licensed cartoon characters or celebrity persons as a background graphic.

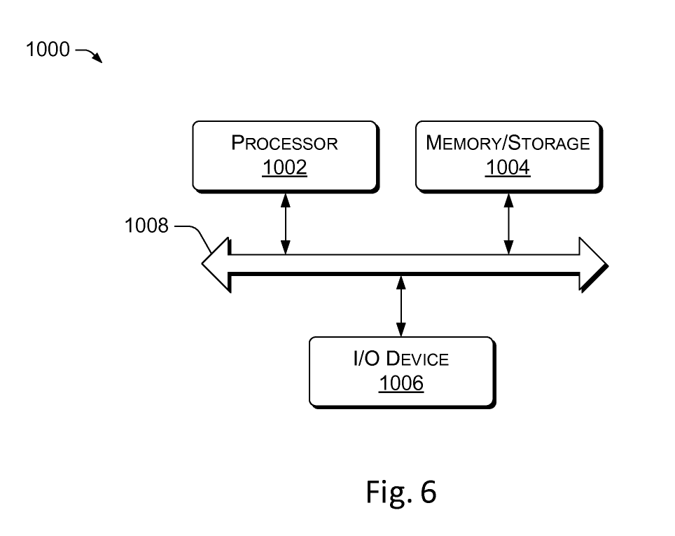

FIG. 6 shows an example architecture for a computing device 1000 or other controller and associated support components that may used with implementations of the example exercise aid 1 or an associated remote control, remote monitoring device, or ancillary system (smartphone, tablet, computer, game system, etc.) accessed by the example exercise aid 1 via Bluetooth or other wireless telemetry.

The example computing device 1000 is only one example and is not intended to suggest any limitation as to scope of use or functionality of the computing device and/or its possible architectures. Neither should computing device 1000 be interpreted as having any dependency or requirement relating to any one or combination of components illustrated in the example computing device 1000.

Computing device 1000 includes one or more processors or processing units 1002, one or more memory and/or storage components 1004, one or more input/output (I/O) devices 1006, and a bus 1008 that allows the various components and devices to communicate with one another. Bus 1008 represents one or more of any of several types of bus structures, including a memory bus or memory controller, a peripheral bus, etc.

Memory/storage component 1004 represents one or more computer storage media. Component 1004 may include volatile media, such as random access memory (RAM), and/or nonvolatile media, such as read-only memory (ROM), flash memory, etc.

One or more input/output devices 1006 may allow a user to enter commands and information to computing device 1000, and also allow information to be presented to the user and/or other components or devices, e.g., via an interface, transmitter, or transceiver.

Software stored and executed may include routines, programs, objects, components, data structures, and so forth that perform particular tasks or implement particular abstract data types. An implementation of these modules and techniques may be stored on or transmitted across some form of computer readable media. Computer readable media can be any available medium or media that can be accessed by a computing device. By way of example, and not limitation, computer readable media may comprise “computer storage media.”

“Computer storage media” include non-volatile, removable and non-removable media implemented in a method or technology for storage of information such as computer readable instructions, data structures, program modules, or other data. Computer storage media include, but are not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

### CONCLUSION

Although exemplary systems and methods have been described in language specific to structural features or techniques, the subject matter defined in the appended claims is not necessarily limited to the specific features or acts described. Rather, the specific features and acts are disclosed as example forms of implementing the claimed systems, methods, and structures.

1. An apparatus, comprising:
a flexible sheet;
a 2-dimensional shape of the flexible sheet formed to intervene between parts of a user and a vertical wall or surface during an exercise of the user against the vertical wall or surface;
a first side of the flexible sheet adherent to the vertical wall or surface; and
a second side of the flexible sheet having a low-friction surface for the exercise of the user against the vertical wall or surface.

2. The apparatus of claim 1, wherein the first side of the flexible sheet comprises an adhesive.

3. The apparatus of claim 1, wherein the first side of the flexible sheet is removably attachable to a surface through a temporary adhesion or a static cling.

4. The apparatus of claim 1, wherein the second side of the flexible sheet comprises a low friction or a slippery plastic surface.

5. The apparatus of claim 1, further comprising an embedded heart monitor.

6. The apparatus of claim 1, further comprising at least one pressure sensor.

7. The apparatus of claim 1, further comprising an embedded processor.

8. The apparatus of claim 1, further comprising an embedded transceiver.

9. The apparatus of claim 8, further comprising a remote in wireless communication with the embedded transceiver.

10. The apparatus of claim 9, wherein the remote provides control of the apparatus.

11. The apparatus of claim 9, wherein the remote displays an audio or a video exercise program communicated by the transceiver.

12. The apparatus of claim 9, wherein the remote monitors a telemetry signal communicated by the transceiver.

13. The apparatus of claim 1, further comprising additional fastening hardware, a screw, a bolt, a nail, a molly bolt, a tape, a staple, or an adhesive.

14. A method, comprising:
making a flexible sheet with an adherent first side for removably fixing to a vertical wall or surface;
making a low-friction second side of the flexible sheet for enabling a user to slide against the flexible sheet during an exercise of the user; and
cutting the flexible sheet into a 2-dimensional shape suitable for intervening between the user and the vertical wall or surface during the exercise.

15. The method of claim 14, further comprising embedding a processor in the flexible sheet to detect a physiological parameter.

16. The method of claim 15, further comprising embedding a heart monitor in the flexible sheet.

17. The method of claim 15, further comprising embedding at least one pressure sensor in the flexible sheet.

18. The method of claim 15, further comprising embedding at least one transceiver in the flexible sheet.

19. An apparatus, comprising:
a plastic sheet;
a low-friction surface of the plastic sheet enabling a user to slide against the plastic sheet without the user adhering to the plastic sheet; and
a microprocessor embedded in the plastic sheet to facilitate an exercise of the user.

20. The apparatus of claim 19, wherein the microprocessor performs a function selected from the group consisting of collecting a physiological parameter of the user, collecting input from a pressure sensor embedded in the plastic sheet, collecting input from a physiological sensor embedded in the plastic sheet, transmitting a telemetry signal, playing an audio exercise program, displaying a video exercise program, receiving commercial programming, transceiving signals with to or from a remote, tranceiving signals with a mobile device, providing real time exercise feedback, providing a programmed exercise routine, and enabling remote monitoring.
